(19) United States
(12) Patent Application Publication
(10) Pub. No.: US 2006/0155250 A1
(43) Pub. Date: Jul. 13, 2006

(54) Dialysis catheter set and method of using same

(76) Inventors: Masahiro Endo, Tokyo (JP); Sobue Katsuyoshi, Tokyo (JP); Minoru Kubota, Yokohama-shi (JP); Atsuyoshi Nomura, Tokyo (JP)

Correspondence Address: BAXTER HEALTHCARE CORPORATION, 1 BAXTER PARKWAY, DF2-2E, DEERFIELD, IL, 60015, US

(21) Appl. No.: 10/535,034

(22) PCT Filed: Dec. 4, 2002
(86) PCT No.: PCT/US02/38760

§ 371 Date: Dec. 16, 2005

(51) Int. Cl. A61M 25/00

(52) U.S. Cl. 604/264; 604/29

(57) ABSTRACT

A catheter set and method of use is provided. The set includes an insert, which fills the majority of the inside annular space within the catheter. Only a small space is left between the catheter and the insert, which is filled with a heparinized saline. The insert and saline prevent fibrin, proteins and other material from collecting inside the catheter, which is especially helpful in the Stepwise Moncrief and Popovich (“SMAP”) catheter insertion, where the catheter is left inside the patient for one to six months before PD therapy begins.

### BACKGROUND OF THE INVENTION

The present invention relates generally to medical treatments. More specifically, the present invention relates to a catheter and method of using same in medical treatments, such as Peritoneal Dialysis (“PD”).

Due to disease or other causes, a person's renal system can fail. In renal failure of any cause, there are several physiological derangements. The balance of water, minerals and the excretion of daily metabolic load is no longer possible in renal failure. During renal failure, toxic end products of nitrogen metabolism (urea, creatinine, uric acid, and others) can accumulate in blood and tissues.

Kidney failure and reduced kidney function have been treated with dialysis. Dialysis removes waste, toxins and excess water from the body that would otherwise have been removed by normal functioning kidneys. Dialysis treatment for replacement of kidney functions is critical to many people because the treatment is life saving. One who has failed kidneys could not continue to live without replacing at least the filtration functions of the kidneys.

PD uses a dialysis solution or dialysate, which is infused into a patient's peritoneal cavity. The dialysate contacts the patient's peritoneal membrane in the peritoneal cavity. Waste, toxins, and excess water pass from the patient's bloodstream through the peritoneal membrane and into the dialysate. The transfer of waste, toxins, and water from the bloodstream into the dialysate occurs by diffusion and osmosis because there is an osmotic gradient across the peritoneal membrane. The spent dialysate is drained from the patient's peritoneal cavity to remove the waste, toxins and water from the patient. New dialysate replaces the spent dialysate and the process is repeated.

During dialysis therapy, a dialysis fluid exchange generally includes draining spent dialysis fluid from the peritoneal cavity and filling the peritoneal cavity with fresh dialysate. Dialysis fluid exchanges have been performed manually (Continuous Ambulatory Peritoneal Dialysis or “CAPD”), usually by the patient, or automatically (Automated Peritoneal Dialysis technique or “APD”), by an automated dialysis machine.

Both the manual and automated PD techniques require insertion of a catheter into the peritoneal cavity of the patient. A dialysis solution (“dialysate”) is introduced through the catheter into the peritoneal cavity of a patient. The dialysate remains in the peritoneal cavity for several hours. Thereafter, the dialysate is removed from the peritoneal cavity carrying with it diffused breakdown products from the blood. The spent dialysate container is disconnected and discarded, wherein a new container of dialysate fluid is attached and the process is repeated. The process is repeated typically several times. The catheter implantation, however, is semi-permanent and a single catheter is used for many PD exchanges.

The installation of the PD catheter can have complications, which despite improvements in catheter implementations over the last few years, can still lead to the removal of the catheter. Catheter related problems can even cause a temporary or permanent transfer to hemodialysis (“HD”) for many patients. The incidence of peritonitis, another major problem with PD, is declining due to the introduction of new connectology, which highlights further the need to address catheter related complications.

The success of PD catheter implantation depends upon the degree of avoidance of various problems such as: inflow/outflow failure, leakage, abdominal wall-related hernias and catheter infection. Leakage is related to the catheter implantation technique, trauma, and/or anatomical abnormalities of the patient. Leakage can occur early (e.g., less than thirty days after implantation), or late (e.g., greater than thirty days), following the start of PD, and can be external or subcutaneous.

Early leakage is usually external, appearing as fluid through the wound or the exit site. Subcutaneous leakage can develop at the site of the incision or at the entry into the peritoneal cavity soon after catheter implantation and after PD commences. Leakage at the exit site or through the wound increases the risk of peritonitis. The cause of late leakage is often difficult to diagnose.

One solution for alleviating the problems caused by leakage is allowing the catheter insertion wounds to heal before beginning PD therapy. Certain techniques for burying PD catheters, such as the Stepwise Moncrief and Popovich (“SMAP”) technique, require a period of time to pass after the catheter resides completely subcutaneously in the patient before PD treatments begin.

Inflow/outflow obstructions occur frequently and can have various causes, such as: (i) mechanical obstruction (tip migration, kinks, etc.); (ii) constipation; and (iii) catheter blockage. Outflow obstructions (typically one-way obstructions) are the most frequent obstructions, causing poor flow and failure to drain the peritoneal cavity. Outflow obstructions can be caused by factors present inside the catheter, such as from debris due to a blood clot or fibrin, or from factors present outside the catheter, such as bowel enwrapping the catheter (constipation), occlusion of the catheter holes, catheter tip dislocation or tip entrapment in peritoneal pockets.

Inflow obstructions are caused by the kinking of the catheter, for example in the subcutaneous tunnel, or again from debris existing inside the catheter such as clots or fibrin. Techniques for removing obstructions range from noninvasive approaches, such as: (i) body position changes and laxatives, (ii) pushing or sucking with a heparinized saline, (iii) infusing fibrinolytic agents (e.g., urokinase, streptokinase) into the catheter for a number of hours; or (iv) heparin in doses. Techniques for removing obstructions also include more aggressive techniques, such as: (i) fluoroscopically guiding a stylet through the catheter; (ii) cleaning the catheter with an intraluminal brush or other utensil; or (iii) using peritonescopy to visually correct or replace the catheter.

Leaving the catheter in the patient's body for a period of time before beginning PD therapy decreases the likelihood of leakage but increases the likelihood that fibrin, waste, proteins or other materials will enter the catheter and form a block. A need exists for an apparatus and method that allow the catheter to reside inside the patient for an extended period of time and to prevent the catheter during this time from becoming partially or completely blocked.

### SUMMARY OF THE INVENTION

The present invention includes an improved dialysis catheter and method of inserting same. One insertion technique useful with the catheter of the present invention, the SMAP technique, is becoming increasingly popular. The SMAP technique allows various types of PD catheters to be implanted into the peritoneal cavity in the usual manner. Afterward, an external portion of the catheter is buried temporarily under the skin. Fifteen centimeters typically of the catheter is directed back through the exit site and under the skin where it remains for a period of time, such as one to six months. With this technique, PD catheters are implanted in advance of the need for their use. The technique reduces leakage by allowing leak points to heal after implantation and also allows a fibrous ingrowth to the catheter cuffs (the cuffs are tubular mounting structures that fix the inserted connector within the patient) to be completed.

While the catheter set and method of implanting same are described in connection with the SMAP method, the catheter of the present invention may have various configurations as described below, may be used with various installation techniques and may be used with various different types of dialysis, such as PD and CAPD. The catheter includes an insert, otherwise termed herein as an obstructor. The obstructor fills the majority of the inside annular space within the catheter. The obstructor is generally tubular in an embodiment and has an open extraperitoneal end and either an open or closed intraperitoneal end.

Only a small space is left between the catheter and the insect, which is filled with saline, such as heparinized saline. The insert and saline prevent fibrin, proteins and other material from collecting inside the catheter, which is especially helpful in the SMAP method where the catheter is left inside the patient for months before PD therapy begins.

To insert the catheter and obstructor, the obstructor is fitted with a stylet, which is stiff relative to the catheter and insert and allows the doctor to position properly the catheter into the peritoneal cavity. The stylet also has a ring or grasping portion extending out of the insert that allows the stylet to be grasped easily. After the doctor has placed the catheter into the patent, the doctor removes the stylet, leaving the catheter.

The intraperitoneal end of the catheter defines a number of apertures that allow dialysate to flow into and out of the catheter from various places inside the peritoneal cavities. The insert or obstructor likewise defines a series of holes, e.g., one to thirty holes. The insect holes are located closer to inlet of the catheter and insert than are the catheter holes, e.g., near the cuffs of the catheter. The insert holes are located alternatively on one side or the other from the center of the insert. After the stylet is removed, the doctor secures the inner cuff to the peritoneal membrane and ensures that the catheter is unobstructed by injecting saline via a syringe. The saline fills the annular cavity of the obstructor. A portion of the saline exists the insert holes and fills the small space between the obstructor and the catheter.

The SMAP method involves the creation of a “swan-neck” type turn with the extraperitoneal portion of the catheter, which requires additional incisions to be made in the patient. To make these incisions, the doctor uses a subcutaneous cutting device known as a trocar. The trocar fits into the extraperitoneal end of the catheter or insert and guides the catheter using the trocar.

After the cuffs are in place and the extraperitoneal portion of the catheter is inserted for its temporary stay inside the patient, the doctor injects heparinized saline into the insert and places a plug in the extraperitoneal end of the obstructor to hold the saline in place and to close the open end of the peritoneal cavity. One or more pieces of surgical string may be necessary lo tighten the catheter/insert/plug assembly. The method of the present invention may be performed alternatively without (he use of surgical string.

The doctor staples and/or sows together the implantation incisions with the catheter buried completely subcutaneously. After the waiting period of four weeks to six months, the doctor makes a different incision to retrieve the external portion. The doctor locates the catheter end by touch typically, however, the insert and plug each contain a radio opaque strip so that the location of the catheter/obstructor can be found by x-ray if needed.

Upon retrieving the external portion of the catheter, the doctor removes the plug and again injects saline into the insert. The saline fills the annular space of the insert and flows out of the insert apertures into the small space between the insert and the catheter. In this manner the saline acts as a lubricant for removing the insert from the catheter. The insert is removed easily. The external end of the catheter is secured to the outside of the patient, who is then ready to receive PD treatment.

It is therefore an advantage of the present invention to provide a catheter set for use with dialysis.

It is another advantage of the present invention to provide a catheter set for use with different types of catheters.

It is a further advantage of the present invention to provide a catheter set for use with different types of catheter implantation procedures.

Moreover, it is an advantage of the present invention to provide a catheter set for use with different types of peritoneal dialysis procedures.

It is still further an advantage of the present invention to provide a catheter set for use with the SMAP insertion method.

It is still another advantage of the present invention to provide a catheter set for dialysis that dissuades fibrin and other contaminants from entering the catheter.

Additional features and advantages of the present invention are described in, and will be apparent from, the following Detailed Description of the Invention and the figures.

### BRIEF DESCRIPTION OF THE FIGURES

FIG. 1 is a schematic perspective view of the various devices of the catheter set of the present invention.

FIGS. 2 to 4 illustrate schematically different configurations for the intraperitoneal portion of the catheter of the present invention.

FIGS. 5 to 13 illustrate schematically different configurations for the extraperitoneal portion of the catheter of the present invention.

### DETAILED DESCRIPTION OF THE INVENTION

The present invention includes a dialysis catheter, a medical set including the catheter and a method of installing same. A number of catheter related complications are associated with PD, including dialysate leak, fibrin plug, outflow obstruction, cuff extrusion, herniation, exit-site or tunnel infection, and peritonitis. Several of these complications may necessitate catheter repositioning and occasionally replacement. Efforts to reduce the catheter-related complications associated with PD have focused on improved connection technology, new implantation techniques and innovative catheter designs.

One approach to reduce leakage has been to delay use of the peritoneal catheter. after insertion to permit complete healing of the subcutaneous tunnel. This method helps to avoid several factors that predispose to the development of exit-site infections, including peritoneal dialysate leaks and excessive pulling and twisting of the catheter during exchanges. One risk associated with delayed initiation of PD following catheter placement is the increased likelihood that the installed catheter will become partially or fully blocked within material, such as fibrin, waste, proteins and other toxins. The purpose of the present invention is to enable the catheter to reside inside the patient for a desired period of time, such as one to six months, before PD therapy begins and at the same time prevent the catheter from becoming blocked or obstructed as well as to make catheter implantation easier.

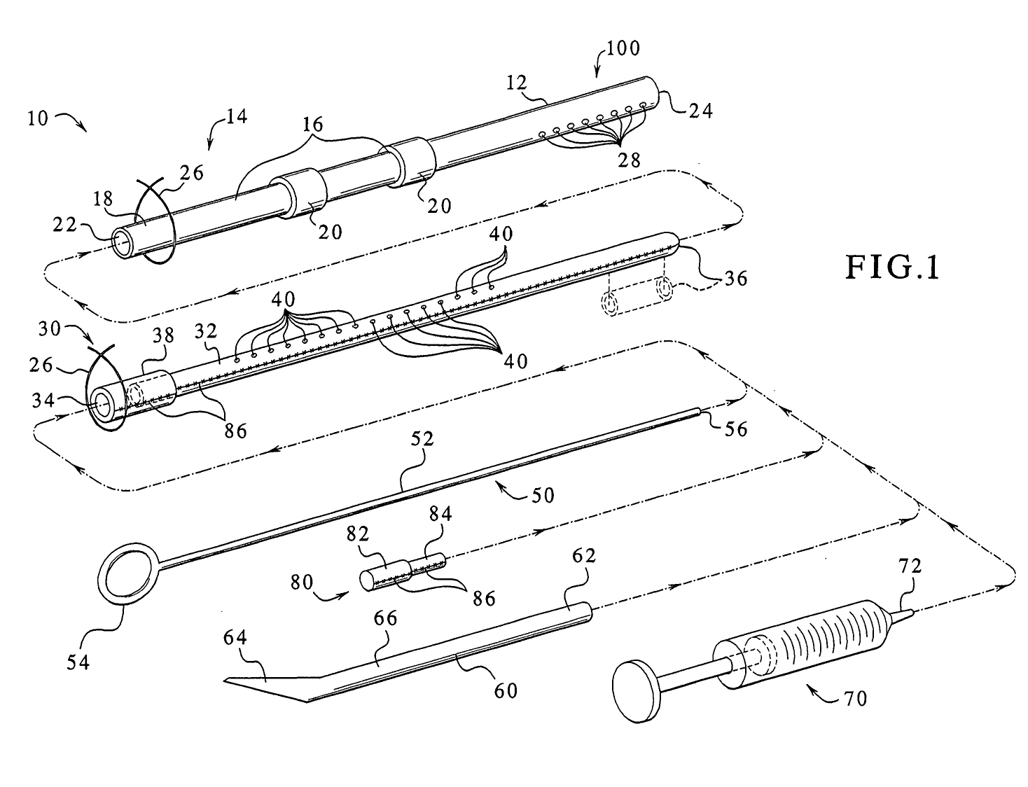

Referring now to the drawings and in particular to FIG. 1, a catheter set 100 is illustrated. The set includes a catheter 10. Catheter 10 includes an intraperitoneal portion 12 and an extraperitoneal portion 14. The extraperitoneal portion 14 includes a subcutaneous part 16 and an external part 18 that resides eventually outside the patient's body, i.e., beyond the exit site.

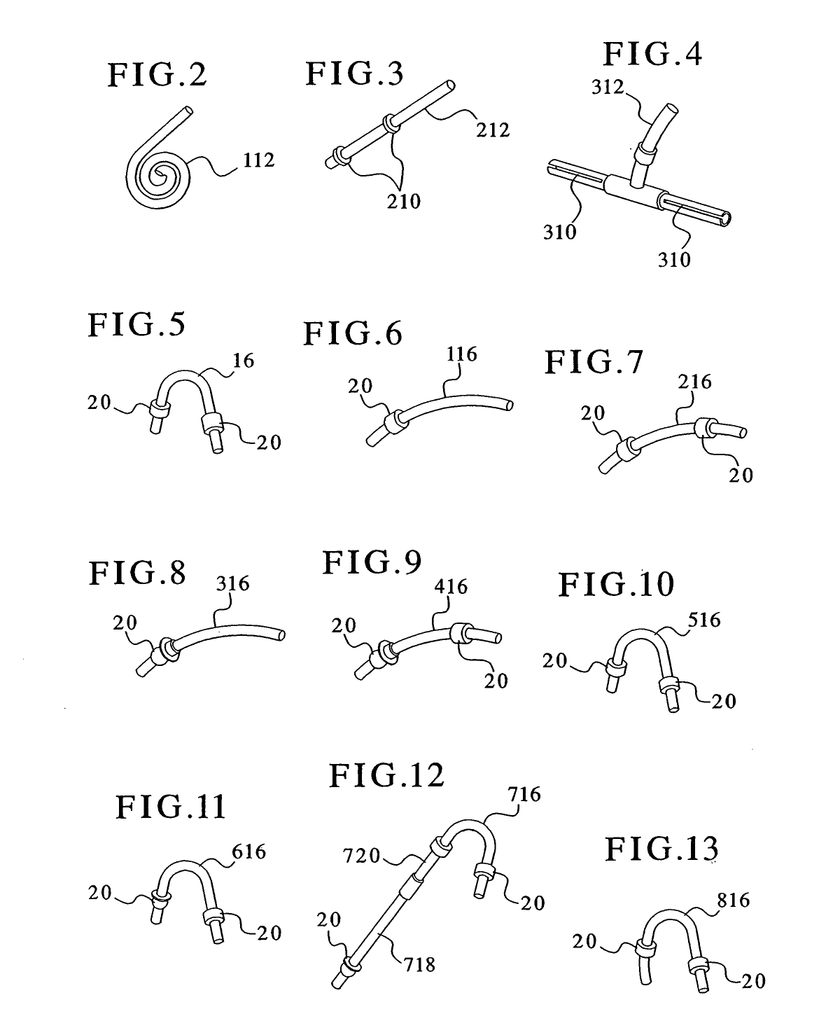

The subcutaneous part 16 of the extraperitoneal portion 14 of the catheter 10 includes at least one cuff 20. The cuffs 20 enable the catheter to be structure or anchored inside the patient's body. FIG. 1 is illustrated with a two-cuff, straight Tenckhoff type catheter 10, which is used widely because it satisfies the needs of many patients. Many catheter variations exist, which are designed to minimize complications of pain, inadequate flow and infection. The present invention expressly includes each of these variations, some of which are illustrated in FIGS. 2 to 13.

FIGS. 2 to 4 illustrate variations for (he intraperitoneal portion 12 of the catheter 10. Besides the straight intraperitoneal portion 12 illustrated above in FIG. 1, portion 112 can be coiled as illustrated in FIG. 2. The coiled portion 112 can be used with most of the extraperitoneal configurations in FIGS. 5 to 13. The coiled configuration 112 provides an increased bulk of tubing and more side holes (not illustrated) for outflow.

Portion 212 of FIG. 3 includes silicone or polymer disks 210. Silicone discs 210 extend perpendicular to the catheter portion 212 and hold the bowels away from the exit holes (not illustrated). Disks 210 also help to minimize catheter tip migration. Portion 312 of FIG. 4 is known as a “T-Flute” catheter. Instead of side holes, portion 312 defines a plurality of thin, longitudinal “flutes” or grooves 310.

Referring now to FIGS. 5 to 13 variations of the subcutaneous parts of the extraperitoneal portion of the catheter of the present invention are illustrated. FIG. 5 illustrates subcutaneous part 16 of portion 14 of FIG. 1 in more detail. The SMAP part 16 includes a swan-neck as illustrated. The external skin cuff 20 is elongated to about 2.5 centimeters and is tapered at the ends.

FIGS. 6 to 9 illustrate straight variations. FIGS. 6 and 7 illustrate straight subcutaneous parts 116 and 216 with single and double cuffs 20, respectively. FIGS. 8 and 9 illustrate straight configurations 316 and 416 with single and double cuffs 20, respectively, wherein the deep cuff 20 has a bead and/or flange. The bead and flange strengthen the anchorage of the catheter to the abdominal wall, which increases the mass of tissue in growth into the cuff/flange structure, decreasing the risk of leakage. The bead and flange can be affixed to the tubing at a forty-five degree angle to orient the intraperitoneal portion 12 properly.

The single cuff subcutaneous parts 116 and 316 have been known to have more exit site complications and shorter survival times than the double-cuff subcutaneous parts 216 and 416, rendering the double cuff type catheters preferable in an embodiment. The cuffs 20 are made of polyester fiber in an embodiment. The distance between two cuffs 20 is about five centimeters in an embodiment.

FIGS. 10 to 13 illustrate various types of swan-neck subcutaneous parts. Swan-necks lessen the occurrence of cuff extensions and catheter tip migration associate with straight catheters. The 90 to 180 degree bend allows the catheter to exit the skin pointing downward and yet enter the peritoneum pointing toward the pelvis, in an unstressed condition.

FIGS. 10 and 11 illustrate subcutaneous parts 516 and 616 with performed bends, eliminating the resilience force or “shape memory” of straight catheters. One of the cuffs 20 of subcutaneous part 616 includes a bead and flange. FIG. 12 illustrates a swan-neck subcutaneous part 716, which has a subcutaneous tunnel of extended length. Subcutaneous part 716 includes two silicone rubber tubes 718 and 720 that are connected at the time of implantation. The implanted lower tube 718 forms the intraperitoneal portion 12 and has one cuff 20 with a bead and flange. The upper tube 720 has two cuffs 20, one on either side of the bent segment. FIG. 13 illustrates a swan-neck subcutaneous part 816, which has two right angle bends, one to direct the intraperitoneal portion 12 parallel to the peritoneal peritoneum, and one to direct the subcutaneous portion 14 downward toward the skin exit site.

Each of the catheters and portion is illustrated in FIGS. 1 to 13 can be made of one or more of a plurality of materials. One common material is smooth silicone rubber. Silicone is biocompatible because it is inert, soft, flexible, and contains no known harmful plasticizers. Another suitable catheter material is polyurethane. Polyurethane provides more wall strength than silicone, allowing for thinner walled catheters with larger lumens. Other suitable materials include teflon, polypropylene as well as other biocompatible materials.

Referring again to FIG. 1, the catheter set 100 includes an insert or obstructor 30, which is fitted into any of the intraperitoneal portions or subcutaneous parts described above in connection with FIGS. 2 to 13, as well as the portions 12 and 14 illustrated in FIG. 1. For purposes of describing the present invention, insert and obstructor mean the same. Insert 30 includes an elongated tubular portion 32 having an open extraperitoneal end 34 and an open or closed intraperitoneal end 36 (end 36 of portion 32 shown in solid as being closed and in phantom as alternatively being open).

Extraperitoneal end 34 includes a larger diameter portion 38. Larger diameter portion 38 in an embodiment is a section of tube that fits snugly over the outer diameter of the elongated tubular portion 32, wherein larger diameter portion 38 is adhered with a medically safe sealant, heat scaled, ultrasonically welded or otherwise connected permanently to the elongated tube portion 32 in such a manner that the method of attachment is safe physiologically to contact internal bodily fluids.

Obstructor 30 can be made from one or more materials. Materials suitable for use with the insert or obstructor include silicone, teflon (hard or soft), polyethlene, polyurethane, any other type of biocompatible plastic and any combination of these. In an embodiment, the insert includes a metal frame, such as a metal mesh or metal spiral, to reinforce the strength of the insert and to make the insert more rigid. The metal used to reinforce the insert is biocompatible, such as stainless steel or titanium.

When catheter 10 is inserted into the patient, the insert 30 resides inside catheter 10 so that intraperitoneal end 36 of insert 30, which is alternatively open or closed, is at or extending past the end 24 of the catheter 10 at the intraperitoneal portion. The larger diameter portion 38 of the extraperitoneal end 34 of insert 30 wedges into, i.e., pressure fits inside end 22 of catheter 10 at the external part 18 of tie extraperitoneal portion 14. Catheter end 22 may be secured to: (i) the larger diameter portion 38, (ii) the elongated tube portion 32 directly adjacent to the larger diameter portion 38, and/or (iii) the intraperitoneal end 36, respectively, via one or more pieces of surgical string 26.

In an alternative embodiment, larger diameter portion 38 of the extraperitoneal end 34 of insert 30 abuts end 22 of catheter 10 rather than press fitting into end 22. In this instance, the outside diameter of larger diameter portion 38 is the same as, i.e., flush with the outside diameter of catheter 10. A third, larger tube section (not illustrated) is then slid over the interface between larger diameter portion 38 and catheter 10. The third tube section is adhered onto portion 38 and catheter 10 via a medically safe sealant or via method discussed above in connection with the connection of portion 38. After retrieving the external part 18 of catheter 10, the doctor removes insert 30 by cutting away the third tube with scissors.

The catheter 10 and insert or obstructor 30 are sized so that the elongated tube portion 32 fits very snugly inside catheter 10. In an embodiment, the inner diameter of catheter 10 is about 3 millimeters, while the outside diameter of the elongated tube portion 32 is about 2.8 millimeters. About one tenth of a millimeter resides therefore between the outer surface of the elongated portion 32 and the inner surface of catheter 10. The larger diameter portion 38 has a diameter of greater than 3 mm, which allows portion 38 to stretch the polymer catheter 10 to make a tight press fit.

The catheter 10 and insert 30 are glided by a doctor collectively using a guide or stylet 50, which is inserted initially into insert 30. The stylet 50 includes a thin rod portion 52. A ring portion or handle 54 is formed at one end of rod portion 52 of stylet 50. The end 56 of stylet 50 is inserted into insert 30 so that end 56 extends all the way to the end 36 of insert 30. Ring 54 resides just outside of the larger diameter portion 38 of insert 30. The guide or stylet 50 is a biocompatible metal in an embodiment, such as stainless steel or titanium.

Stylet 50 enables the doctor to grab and grasp ring 54 and provides enough rigidity in combination with the insert 30 and catheter 10 to guide the intraperitoneal portion 12 deep into the patient's peritoneal cavity. The doctor guides end 56, intraperitoneal end 36 of insert 30, which is open or closed, and end 24 of catheter 10 collectively so that at least a portion of the apertures 28 of catheter 10 reside along the bottom of the patient's peritoneal cavity.

After the catheter 10, insert 30 and stylet 50 have been inserted into the proper position within the patient, the doctor removes stylet 50. Depending on the type of extraperitoneal design of the catheter, there exists a length of the catheter, namely, the subcutaneous part 16 and external part 18 that remain outside the patient's body even after the catheter assembly is inserted into the peritoneal cavity. The doctor holds a section of the portion residing outside the patient's body and removes easily the guide or stylet 50 from inside of the insert 30. The larger diameter portion holds insert 30 snugly within the catheter 10, despite any shearing force created while the stylet 50 is being removed from the elongated tube portion 32 of the insert 30. The catheter 10 and the obstructor 30 remain in the proper implanted position within the peritoneal cavity during and after the removal of the guide or stylet 50 from insert 30.

After removal of stylet 50, a trocar 60 is secured to the extraperitoneal end 34 of the insert 30. Trocar 60 is a cutting instrument made of a biocompatibly safe metal, such as stainless steel or titanium. Trocar 60 includes attachment end 62 and a blade end 64. Attachment end 62 is inserted into either the larger diameter portion 38 or the elongated tube portion 32 of insert 30, wherein surgical string 26 is tied about portion 38 or the catheter 10 to secure catheter 10 and insert 30 to trocar 60.

As is well known in the art, the doctor uses the trocar 60, which includes an extended handle 66, to insert blade 64 into the original incision made in the patient and cut a second incision in the patient from underneath the skin, i.e., subcutaneously. The doctor then installs the extraperitoneal portion 14 of catheter 10 according to the desired implementation. As discussed above, in one preferred embodiment, the set 100 is used for the SMAP method of implantation. With SMAP, or any procedure requiring a swan-neck type of bend, the doctor reinserts the trocar through the second incision, cuts a thin incision subcutaneously using the trocar 60, bends and moves the catheter inside the patient and then removes the trocar from the extraperitoneal end 34 of insert 30 and likewise from catheter 10.

The doctor then uses a syringe 70 to inject a heparinized saline into the end 34 and cavity defined by insert 30. The syringe 70 includes an injection end 72 that fits either inside of the larger diameter portion 38 or inside of the elongated tube portion 32 of the insert 30. The injected the heparinized saline performs two functions. First, the saline fills the inner cavity defined by the elongated tube portion 32 of insert 30 with liquid, further preventing fibrin, proteins, etc. from entering the insert 30, e.g., through apertures 40. Second, the small volume residing between the outer surface of the elongated portion 32 and the inner surface of the catheter 10 is filled with the heparinized saline via the apertures 40 defined by elongated portion 32. The heparinized saline helps to keep fibrin and proteins, etc., from entering the catheter 10 through apertures 28 or end 24.

Insert 30 of set 100 does not have to define apertures 40. In one preferred embodiment, however, at least one and as many as thirty apertures 40 are defined by elongated tube portion 32. Apertures 40, as with apertures 28 in catheter 10, may reside on opposite sides of the elongated portion 32, for example, as pairs, or be one more very thin flutes. The apertures 40 in one preferred embodiment are located at a different portion of the length of insert 30 than are apertures 28 along a corresponding length of catheter 10. For example, as illustrated, the apertures 28 of catheter 10 are located closer to end 24 of catheter 10, while apertures 40 in the elongated tube portion 32 are located closer to the extraperitoneal end 34, e.g., corresponding to approximately the cuff 20 closest to end 22 of catheter 10.

For SMAP as well as other types of PD, the subcutaneous part 16 makes a swan-neck 90 to 180 degree bend, so that end 22 of catheter 10 and extraperitoneal end 34 of insert 30 point upwards toward the patient's head. The second incision enables the doctor to bend the catheter/insert to make the desired “U” turn. The third incision enables the doctor to remove the end 22 of catheter 10 and extraperitoneal end 34 of insert 30 to: (i) remove the trocar from the larger portion 38 or end 34 of insert 30; (ii) insert saline via syringe 70 as described above; and (iii) insert a plug 80 into larger diameter 38 or elongated portion 32 to hold the saline with insert 30.

Plug 80 in an embodiment includes a larger diameter section 82 that fits snugly into larger diameter portion 38 and a smaller diameter portion 84 that fits snugly into elongated portion 32. The larger diameter section 82 can extend out from larger diameter portion 38 or include a head that enables the doctor to grasp and remove plug 80 after it has been inserted into obstructor 30. One or more pieces of surgical string 26 can be used about catheter 10 or portion 38 to tighten and help seal plug 80. Plug 80 and insert 30 each include a radio opaque strip 86 that enables insert 30 to appear on an x-ray.

Once implanted, the doctor sows or staples closed the three incisions. Thereafter, the doctor should be able to locate external part 18 by touch. Radio opaque strip 86 is safeguard against movement of extraperitoneal portion 14 and part 18 or in cases of extreme obesity.

For SMAP, the catheter is left inside the patient for a period of time, such as four weeks to six months during which PD therapy is not performed. This dwell time allows tissue to grow around the cuffs and the implantation wounds to heal, decreasing the risk of leakage. After this period of time, the doctor removes the external part 18 of the extraperitoneal portion 14 of the catheter 10 from the body so that the catheter can be used for therapy. The doctor determines an exit site for the external part 18, which is usually either above or below but not on the belt line, not on a scar (i.e., at any of the locations for inserting the catheter 10) and not in abdominal folds.

Once the doctor has located and removed the external part 18 of catheter 10, the doctor removes plug 80 from insert 30. To remove insert 30, the doctor again injects saline via syringe 70 into the end 34 of the insert 30. The injected saline again performs dual functions. First, the saline fills the inner cavity defined by the elongated tube portion 32 of insert 30 with liquid and, trough apertures 40, fills the small volume residing between the outer surface of the elongated portion 32 and the inner surface of the catheter 10 with the saline. The saline flushes built up fibrin and proteins from catheter 10 through apertures 28 and end 24. Second, the saline acts as a lubricant so that insert 30 is thereafter removed easily from catheter 10.

Once insert 30 is removed, the doctor can flush the open catheter with saline. The external part 18 is then secured so that a portion thereof extends outside the body, allowing the patient to begin PD therapy. The insert, taking up much of the lumen of catheter 10 reduces the amount of fibrin and other materials described above from collecting inside catheter 10, increasing the likelihood that the implantation of catheter 10 is successful.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A catheter set for peritoneal dialysis comprising:
a catheter having first and second ends; and
an insert filling a majority of an interior space defined by the catheter, the insert defining a cavity and having an extraperitoneal end and an intraperitoneal end.

2. The set of claim 1, which includes a plug placed in the extraperitoneal end of the insert after the catheter and insert have been implanted in a patient.

3. The set of claim 1, wherein the intraperitoneal end of the insert is open or closed.

4. The set of claim 1, wherein the catheter and insert are packaged together.

5. The set of claim 1, which includes a guide placed in the cavity of the insert before the catheter and insert have been implanted in the patient.

6. The set of claim 1, wherein at least one of the insert and the plug includes a radio opaque member.

7. The set of claim 1, wherein the insert is metal reinforced.

8. The set of claim 1, wherein the catheter is a tube and insert fills most of an open space defined by the tube.

9. The set of claim 1, which includes a syringe for injecting a liquid into the cavity.

10. The set of claim 9, wherein the liquid is saline.

11. The set of claim 1, wherein at least one of the catheter and the insert includes a tubular wall that defines at least one aperture.

12. The set of claim 11, wherein the apertures are located at a different section of the tubular wall of the catheter than are the apertures in the tubular wall of the insert.

13. The set of claim 11, wherein at least one aperture is an elongated flute.

14. The set of claim 1, wherein the insert is at least as long as the catheter.

15. The set of claim 1, which includes at least one piece of surgical string securing the catheter and the insert.

16. A catheter set for peritoneal dialysis comprising:
a catheter leaving first and second open ends;
an insert placed inside the catheter, the insert defining a cavity and having an extraperitoneal end and an intraperitoneal end; and
a guide placed in the cavity and extending from the extraperitoneal end of the insert.

17. The catheter set of claim 16, wherein the guide is metal.

18. The catheter set of claim 16, which includes a trocar secured to the extraperitoneal end of the insert when the guide has been removed.

19. The catheter set of claim 16, wherein the guide defines a portion configured and arranged to be grasped and moved by a person.

20. The catheter set of claim 16, wherein the catheter includes at least one cuff.

21. The catheter set of claim 20, wherein the cuff includes at least one of a bend and a flange.

22. The catheter set or claim 16, wherein a portion of the catheter and insert is coiled.

23. An obstructor for occupying space within a tubular catheter when inserted into a catheter, the catheter defining first and second ends, the obstructor comprising:
a tube including an extraperitoneal end and an intraperitoneal end, the extraperitoneal end including a portion having an increased diameter, the portion contacting the first end of the catheter when the tube is inserted into the catheter, the intraperitoneal end of the tube extending at least substantially to the second end of the catheter, an outer diameter of the tube fitting substantially an interior space defined by the tubular catheter.

24. The obstructor of claim 23, wherein the larger diameter portion is sized to press-fit inside the catheter.

25. The obstructor of claim 23, wherein the tube houses a radio opaque member.

26. The obstructor of claim 23, wherein the tube is a first tube and the larger diameter portion is a second tube adhered to the first tube.

27. The obstructor of claim 23, wherein the tube is made from at least one material selected from the group consisting of silicone, teflon, polyurethane, polypropylene, metal mesh, metal spiral, and an combination thereof.

28. The obstructor of claim 23, wherein the tube defines at least one aperture along its length that allows fluid injected into the extraperitoneal end to exit the tube.

29. The obstructor of claim 23, which includes a plug inserted into the extraperitoneal end.

30. The obstructor of claim 29, wherein the plug is made from at least one material selected from the group consisting of: silicone, teflon, polyurethane, polypropylene, metal and any combination thereof.

31. A method for inserting a catheter comprising the steps of:
making an incision into a patient;
inserting the catheter, the catheter having an insert filling most of the internal space defined by the catheter; and
removing the insert after the catheter has been implanted.

32. The method of claim 31, wherein a guide is placed initially into the insert; and which includes the steps of using the guide to maneuver the catheter inside the patient and then removing the guide.

33. The method of claim 31, wherein the insert includes a tubular length and at least one hole defined by the tubular length, and which includes the step of injecting fluid into the tubular length, through the hole between the insert and the catheter.

34. The method of claim 33, which includes the step of placing a plug in the insert after injecting the fluid into the insert.

35. The method of claim 33, which includes the step of removing the insert after injecting the fluid.

36. The method of claim 31, wherein at least one cuff is positioned on the catheter, and which includes the step of securing the cuff to the patient.

37. The method of claim 31, which includes making a plurality of incisions into the patient and the step of using the multiple incisions to guide the catheter in a plurality of directions.

38. The method of claim 37, which includes the step of using an instrument to bend the catheter inside the patient.

39. The method of claim 31, which includes the step before removing the insert of closing the insertion with the catheter and insert implanted inside the patient.

40. The method of claim 31, wherein removing the insert occurs after a period of time in which the insert and catheter have resided inside the patient.

41. The method of claim 31, which includes the step of flushing the catheter after the insert has been removed.

42. The method of claim 31, which includes the step of securing one end of the catheter to reside outside the body of the patient after the insert has been removed.

43. The method of claim 31, wherein the catheter is preformed to have a bend, wherein inserting the catheter includes inserting the preformed catheter.

44. A method of implanting a catheter comprising the steps of:
implanting the catheter inside a patient, the catheter having an obstruction tending to disallow material from entering the catheter;
leaving the entire catheter inside the patient for a period of time; and
removing the obstruction and securing a portion of the catheter to extend outside the patent.
